(19) United States
(12) Patent Application Publication
(10) Pub. No.: US 2009/0203970 A1
(43) Pub. Date: Aug. 13, 2009

(54) SLEEP APNEA TEST SENSOR ASSEMBLY AND SLEEP APNEA TEST EQUIPMENT USING THE SAME

(75) Inventors: Shogo Fukushima, Moriguch-shi (JP); Matsuki Yamamoto, Ashiya-shi (JP)

Correspondence Address: GREENBLUM & BERNSTEIN, P.L.C., 1950 ROLAND CLARKE PLACE, RESTON, VA, 20191, US

(73) Assignee: MATSUSHITA ELECTRIC WORKS, LTD., Osaka (JP)

(21) Appl. No.: 11/914,596

(22) PCT Filed: May 19, 2006
(86) PCT No.: PCT/JP2006/310024

§ 371 Date: Nov. 16, 2007

(30) Foreign Application Priority Data
May 19, 2005 (JP) 2005-147038

(51) Int. Cl. A61B 5/08

(52) U.S. Cl. 600/301

(57) ABSTRACT

This relates to a sleep apnea test sensor assembly (20) comprising multiple sensors to be used for diagnosis of sleep apnea syndrome, and a test equipment (30) comprising the same. The respective sensors each associate sensing data measured by a respective sensing section with time data of a clock unit, and store them in a storage unit so as to allow the sensing section to perform sensing at a predetermined period in response to a clocking operation of the clock unit, and set time of the clock unit in response to a synchronous signal input from outside. The respective sensors operate physically independently of each other, but can measure sensing data with the synchronization in time therebetween being secured, enabling accurate diagnosis of SAS.

### TECHNICAL FIELD

The present invention relates to a sleep apnea test sensor assembly comprising multiple sensors to be used for diagnosis of sleep apnea syndrome, and a sleep apnea test equipment using the same.

### BACKGROUND ART

Sleep apnea syndrome (SAS) is considered to be one of the sleep disorders. Generally, a sleep polysomnography test is used for diagnosis of this SAS. This test uses a sleep apnea test equipment having many bioinstrumentation sensors such as e.g. a snore sensor, an oronasal airflow sensor, an arterial blood oxygen saturation sensor, a chest movement sensor, a stomach movement sensor, and so on. In this sleep polysomnography test, sensing data from each sensor is stored in a data recorder connected to each sensor. Then, a professional such as a doctor diagnoses SAS by analyzing the features of the change rates of these sensing data and the correlations between the data. The diagnosis accuracy of SAS is expected to increase by using multiple sensors as in the equipment described above.

Further, the diagnosis of SAS is also made by a screening test (simplified sleep polysomnography) which an examinee can do at home. This test uses a test equipment disclosed in e.g. Japanese Laid-open Patent Publication Hei 5-200031. This test equipment is formed of multiple sensors and a data recorder e.g. with a built-in signal processing circuit. These multiple sensors have a sensing section reduced in size, while the data recorder has a size allowing it to be attached to the waist of the examinee. Because of this arrangement, the test equipment disclosed in the patent document described above eliminates the need for time-consuming hospitalization for tests, and makes it possible for the examinee to collect sensing data at home.

Generally, in this screening test, a medical institution lends a test equipment to an examinee. The examinee attaches sensors of the test equipment to the body, and collects sensing data while sleeping one night. The examinee brings this test equipment to the medical institution at a later date, and receives the analysis of the sensing data and the diagnosis of SAS of a professional.

### DISCLOSURE OF THE INVENTION

However, in a conventional test equipment, many sensors are connected to the data recorder by long wires, so that the wires become more complicated with an increase in the number of sensors which causes an increase in the number of wires. Thus, if the examinee is not well trained with the test equipment, there is a risk of not noticing wire misconnection or wire falling off, thereby lowering the reliability of the obtained sensing data. Further, when the posture of the examinee changes during sleep, there is a risk that a sensor may come off the body of the examinee, or a wire may fall off the data recorder, thereby causing the data to be unstable. If the posture of the examinee is restricted to prevent the wire falling off, there is a risk of disturbing the sleep of the examinee. Such a problem does not exist in a sleep polysomnography test done in a medical institution, because a person in charge of the test properly handles it. However, in the screening test done by the examinee at home, it is not possible to properly handle e.g. the wire falling off because the examinee itself is not well trained with the test equipment.

In order to solve the problem described above, a possible method may be to install a transmitter in a sensor for wireless data transmission to the data recorder, thereby removing the wire of the sensor. However, there is a risk that an obstacle to block a wireless signal may be present in the bedroom of the home of the examinee, and that depending on the posture of the sleeping examinee, the wireless signal transmitted from the sensor may not be stably received by the data recorder. Furthermore, the sensor attached to the body becomes more likely to come off the body if the transmission power is increased to stabilize the data transmission, which causes an increase in the size of a battery installed in the sensor and an increase in the weight of the sensor.

Another possible method may be to allow each sensor to have a built-in recorder, and allow the recorder to record measured data so as to extract the data after the test. This method improves the complexity of wires because each sensor operates physically independently. However, according to this method, it is difficult to synchronize the time relationship between the respective sensors to match each other. If the time relationship of the respective sensors is not synchronized, it is not possible to compare the correlation between the measured data of the respective sensors, so that accurate data analysis and diagnosis of SAS cannot be expected. Generally, for the diagnosis of SAS, the occurrence frequency of apneas of at least 10 seconds is counted. Thus, in the screening test to comprehensively analyze the measured data for the diagnosis of SAS, an error of about 10 seconds between the respective sensors exerts a fatal influence. For example, if the measured values of a chest movement sensor and a stomach movement sensor rise and fall at the same time, it is determined as normal breathing. However, if the measured values of the two sensors rise and fall in opposite phases to each other, it is determined that the diaphragm is moving without inhalation. For example, if one period is 5 seconds at 12 (times/minute) breaths, an offset of 2.5 seconds between the data causes the diagnosis result to be reversed.

The present invention solves the above-described problems, and its object is to provide a sleep apnea test sensor assembly and a sleep apnea test equipment comprising this sensor assembly that eliminates the complexity of wires connected to multiple sensors, and allows the multiple sensors to operate independently of each other, thereby reducing the burden on a patient to attach sensors and preventing the sensors from falling off, and that efficiently secures synchronization between the sensing data of the respective sensors, enabling accurate diagnosis of SAS, without using transmission means such as e.g. wires and wireless.

The present invention provides a sleep apnea test sensor assembly comprising multiple sensors to be used for diagnosis of sleep apnea syndrome, wherein: the multiple sensors each comprise: a sensing section; a clock unit; a storage unit for storing sensing data measured by the sensing section and associated with time data of the clock unit; and a control unit for allowing the sensing section to perform sensing at a predetermined period in response to a clocking operation of the clock unit, and for setting time of the clock unit in response to a synchronous signal input from outside; and the multiple sensors are comprised of a combination of at least two of a temperature sensor for measuring nose breath, a temperature sensor for measuring mouth breath, an acoustic sensor for measuring snore, a light sensor for measuring blood oxygen concentration, and acceleration sensors for measuring chest and stomach movements.

According to the structure described above, the multiple sensors forming the sensor assembly each comprise a storage unit for sensing data, and operate independently without wires, so that multiple long wires from outside as in a conventional test equipment are not necessary, thereby reducing the complexity of wires. This makes it possible to provide an examinee with good wearing comfort, and to effectively prevent a sensor from falling off even when the examinee moves its posture during sleep (during test).

Further, the multiple sensors each set the time of the clock unit in response to a synchronous signal input from outside, and each allow the corresponding sensing section to perform sensing at a predetermined period in response to the clocking operation of the clock unit. Thus, even when operating independently, the respective sensors can measure sensing data with the synchronization in time being secured, making it possible to obtain sensing data for accurate diagnosis of SAS. Further, the temperature sensor for measuring nose breath, the temperature sensor for measuring mouth breath and the acoustic sensor for measuring snore are attached near the face of the examinee, so that it can provide good wearing comfort because of the absence of long wires. Even when the examinee changes its posture, the acceleration sensors for measuring the chest and stomach movements are unlikely to come off the body because of the absence of wires.

Furthermore, the present invention provides a sleep apnea test equipment to be used for diagnosis of sleep apnea syndrome, which comprises: a sleep apnea test sensor assembly according to claim 1; and a main apparatus having a storage section for storing the multiple sensors forming the sensor assembly, and a control unit for outputting a synchronous signal to control time of the multiple sensors, wherein the main apparatus collects the sensing data, together with the time data, of the respective sensors while stored in the storage section.

The structure described above sets the time of the multiple sensors all together while stored in the main apparatus. Thus, it provides convenience, and even when operating independently of each other, the multiple sensors can measure sensing data with the synchronization in time therebetween being secured, enabling accurate diagnosis of SAS. Further, the main apparatus can effectively collect the respective sensing data of the multiple sensors together with the time data. This collection of data can be performed by connecting the main apparatus to some sensing data analyzing means such as a personal computer.

### BRIEF DESCRIPTION OF DRAWINGS

FIG. 1 is a block diagram showing a schematic structure of a sleep apnea test sensor assembly and a sleep apnea test equipment according to an embodiment of the present invention;

FIG. 2 is a view showing an arrangement of the sensor assembly and the test equipment when sensing sleep data;

FIG. 3 is a block diagram showing the structure of the sensor assembly and the test equipment;

FIG. 4(a) is a perspective view showing an appearance of the sensor assembly and the test equipment when the sensor assembly is stored in a main apparatus, while FIG. 4(b) is a perspective view showing an appearance of the test equipment;

FIG. 5(a) is a view showing one form of attaching, to an examinee, a temperature sensor for measuring an amount of nose breath, a temperature sensor for measuring an amount of mouth breath and an acoustic sensor for measuring snore, while FIG. 5(b) is a view showing another form;

FIG. 6(a) is a view showing one form of attaching a light sensor for measuring blood oxygen concentration to the examinee, while FIG. 6(b) is a view showing another form;

FIG. 7 is graphs showing test results of a screening test using the test equipment; and

FIG. 8 is graphs showing test results of a screening test using the test equipment.

### BEST MODE FOR CARRYING OUT THE INVENTION

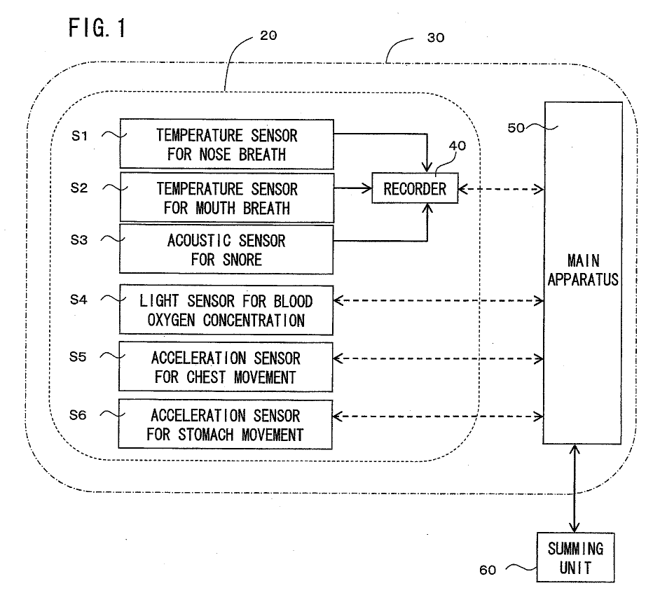

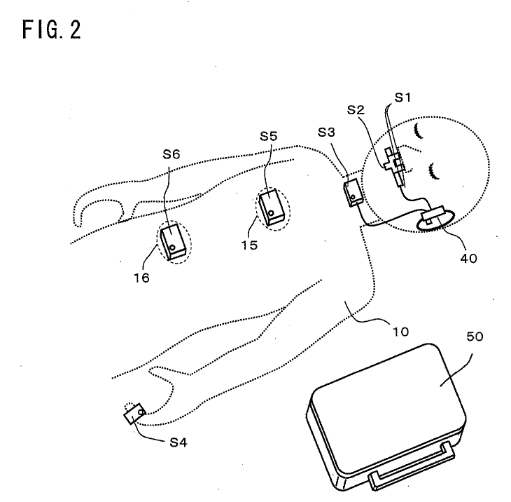

A schematic configuration of a sleep apnea test sensor assembly (hereafter referred to as sensor assembly) and a sleep apnea test equipment comprising this sensor assembly according to an embodiment of the present invention will be described with reference to FIG. 1 and FIG. 2. A sensor assembly 20 of the present embodiment comprises multiple sensors comprised of an appropriate combination of at least two of a temperature sensor S1 for measuring mouth (sic, correctly: nose) breath, a temperature sensor S2 for measuring nose (sic, correctly: mouth) breath, an acoustic sensor S3 for measuring snore, a light sensor S4 for measuring blood oxygen concentration, an acceleration sensor S5 for measuring chest movement, and an acceleration sensor S6 for measuring stomach movement.

The sensor assembly 20 enables more accurate diagnosis of SAS with an increase in the number of kinds of sensors to be combined, but can be put to practical use if at least two kinds of them are combined, without always requiring the use of all kinds of sensors. If there are few kinds of sensors to be combined, such as, for example, if two kinds of sensors are selected, it is preferred that they be selected as follows: That is, if one sensor is selected from sensors placed adjacent to each other, then this one is taken as one kind, while the other kind is selected from the other sensors. More specifically, the sensors S1 to S3 are attached near the head of an examinee 10 to measure data on the breath, whereas the sensors S5, S6 are attached to the body to measure its movement. Thus, if one of the sensors S5, S6 is selected, this one is taken as one kind, while the other kind is to be selected from the sensors other than these.

Among the multiple sensors forming the sensor assembly 20, when measuring, the sensors S1, S2 are attached to a portion under the nose of the examinee 10, the sensor S3 to a portion of the throat of the examinee 10, the sensor S4 to a finger tip of the examinee 10, the sensor S5 to a chest portion 15 of the examinee 10, and the sensor S6 near a stomach portion 16. Further, the sensors S1 to S3, which are attached near the head of the examinee 10, are connected by wires L1, L3 to a recorder 40 for storing sensing data measured thereby. The sensing data measured by the sensors S1 to S3 are once stored in the recorder 40, so that the sensors S1 to S3 including the recorder 40 correspond to the claimed sensors.

A sleep apnea test equipment 30 (hereafter referred to as test equipment) of the present embodiment comprises the sensor assembly 20 described above and a main apparatus 50 for storing the multiple sensors forming the sensor assembly 20 and for communicating with the sensors while stored therein so as to collect sensing data of each sensor with time data. As will be described in detail later, the main apparatus 50 has a storage section for storing the multiple sensors, and has a function to output a synchronous signal to control the time of each sensor while stored therein. After measurement, the main apparatus 50 is connected to a summing unit 60 formed e.g. of a personal computer so as to collect the sensing data from the sensors S1 to S3 via the recorder 40, while directly collecting the sensing data from the sensors S4 to S6, and to transfer them to the summing unit 60.

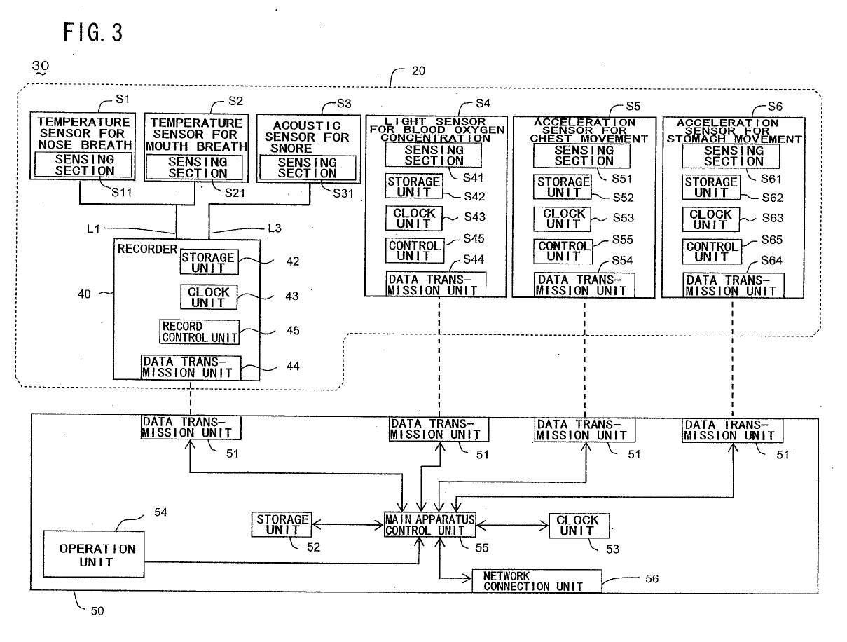

Next, a specific structure of the sensor assembly 20 and the test equipment 30 will be described with reference to FIG. 3. Among the multiple sensors forming the sensor assembly 20, the sensors S1 to S3 comprise sensing sections S11, S21, S31, respectively, without a built-in battery or storage unit. Further, the recorder 40 comprises a storage unit 42 for storing sensing data measured by the sensors S1 to S3, a clock unit 43 for measuring time, a data transmission unit 44, a record control unit 45 for controlling these respective units, and a battery (not shown).

The sensors S1 and S2 are formed of a general purpose temperature sensor, which measures the amount of breath from a temperature change due to the passing of breath. Note that the sensor S1 can separately measure breaths through the left and right nostrils. Further, the sensor S3 uses a small general purpose microphone. The record control unit 45 comprises e.g. a microcomputer, which sets the time of the clock unit 43 in response to a synchronous signal input from the data transmission unit 44, and allows the sensors S1 to S3 to perform sensing at a predetermined period in response to the clocking operation of the clock unit 43.

The sensors S4 to S6 respectively comprise sensing sections S41, S51, S61, storage units S42, S52, S62, clock units S43, S53, S63, data transmission units S44, S54, S64, control units S45, S55, S65 for controlling these respective units, and batteries (not shown).

The sensor S4 is formed of a general purpose light sensor, which allows red and infrared light to penetrate a tip of a finger so as to measure blood oxygen concentration by a difference in absorbance between hemoglobin and oxyhemoglobin in flowing blood. Further, the sensors S5 and S6 are general purpose acceleration sensors which measure three-dimensional acceleration components.

Similarly as in the above-described record control unit 45, the control units S45, S55, S65 set the time of the clock units S43, S53, S63 in response to a synchronous signal input from the data transmission units S44, S54, S64, and allow the respective sensing sections of the sensors S1 (sic, correctly S4) to S6 to perform sensing at a predetermined period in response to the clocking operation of the clock units S43, S53, S63.

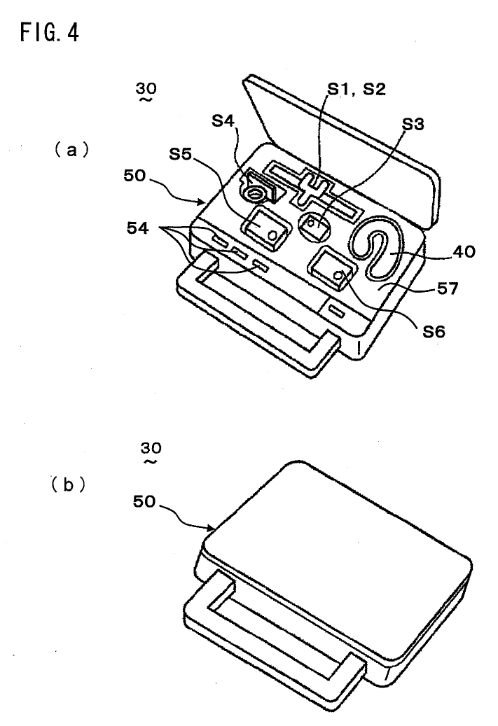

The main apparatus 50 comprises: data transmission units 51 for data transmission while each sensor is stored therein; a storage unit 52 for storing received data; a clock unit 53; an operation unit 54; a main apparatus control unit 55 for controlling the respective units; a network connection unit 56; a storage section 57 (refer to FIG. 4) for storing the multiple sensors forming the sensor assembly 20; and a battery (not shown). When a user inputs an operation command from the operation unit 54, the main apparatus 50 outputs, based on the time of the clock unit 53 via the data transmission units 51, a synchronous signal for controlling the time of the sensors to the sensors while stored therein.

A means for transmission of data between the main apparatus 50 and the multiple sensors of the sensor assembly 20 is not particularly limited, but, for example, has electrodes to allow the data transmission units 51 of the main apparatus 50 to contact the data transmission units 44, S44, S54, S64, respectively, when the multiple sensors are stored in the main apparatus 50, in which data are sent and received through the electrodes using electrical signals.

In a medical institution, the main apparatus 50 is connected to the summing unit 60 via the network connection unit 56. Before measurement, time data is sent from the summing unit 60 to the main apparatus 50 so as to set the time of the clock unit 53. Further, after measurement, sensing data and time data collected in the storage unit 52 of the main apparatus 50 are transferred to the summing unit 60. A general purpose data port is used to connect between the network connection unit 56 and the summing unit 60, in which it is desirable to use a parallel port connection allowing transmission of multiple sensing data and time data at the same time. Otherwise, it is also possible to use a serial port connection such as USB (Universal Serial Bus).

Next, a specific structure of the sensor assembly 20 and the test equipment 30 as well as a process of a sleep apnea test using the test equipment 30 will be described with reference to FIG. 4 to FIG. 6 in addition to the above-described drawings. As shown in FIG. 4(a), before measurement, the sensors S1 to S6 forming the sensor assembly 20 and the recorder 40 are stored in the storage section 57 of the main apparatus 50. This storage section 57 is formed to fit the respective shapes of the sensors S1 to S6 and the recorder 40. Further, as shown in FIG. 4(b), the main apparatus 50 is formed in a bag shape so as to be easily portable with the sensors S1 to S6 and the recorder 40 being stored in the storage section 57.

Before measurement, an examinee synchronizes the time of the respective clock units of the recorder 40 and the sensors S4 to S6. This synchronization is performed by the examinee operating the operation unit 54 while the recorder 40 and the sensors S4 to S6 are stored in the main apparatus 50. Note that when the main apparatus 50 is connected to the summing unit 60, it is also possible for a doctor or the like to operate the main apparatus 50 or the summing unit 60 in advance so as to synchronize the respective clock units.

When receiving a synchronization command based on the operation of the operation unit 54, the main apparatus control unit 55 outputs a synchronous signal based on the time of the clock unit 53 to the recorder 40 and the sensors S4 to S6. The record control unit 45 of the recorder 40 and the respective control units of the multiple sensors forming the sensor assembly 20, when having the synchronous signal input thereto, set the time of the clock units corresponding thereto, respectively, in response to the synchronous signal. This synchronous signal is not necessarily absolutely at an accurate time, and it is sufficient if the respective clock units of the recorder 40 and the sensors S4 to S6 are set to be at the same time. Further, the synchronous signal can not only be time data, but also be a mere trigger, and it is sufficient if the respective clock units are set to be at a base time corresponding to the trigger.

For the synchronization, it is not necessary that all of the recorder 40 and the sensors S4 to S6 are simultaneously stored in the main apparatus 50. For example, at one time point, only the recorder 40 is connected to the main apparatus 50, and the recorder 40 is set to be at time A, while at another time point thereafter, the sensor S4 is stored in the main apparatus 50, and the sensor S4 is set to be at time B. In this example, even if the recorder 40 and the sensor S4 are set to be at time A and time B, respectively, which are different in time, these times are both based on the time of the clock unit 53 of the main apparatus 50. Accordingly, unless the time of the clock unit 53 is changed after the setting at time A and before the setting at time B, the respective clock units of the recorder 40 and the sensor S4 are set to be at the same time. According to this method, even if there are many sensors, it is possible for a user to store multiple sensors in the main apparatus 50 and operate the operation unit 54 so as to efficiently synchronize the time of all the sensors all together.

According to the method described above, the time synchronization of the recorder 40 and the sensors S4 to S6 is achieved without using communication means such as e.g. wires and wireless communication. This synchronization is secured within at least a range of inherent variation of each clock unit. The synchronized recorder 40 and sensors S4 to S6 control the time based on the time of the respective clock units.

Further, even while the recorder 40 and the sensors S4 to S6 are not stored in the main apparatus 50, it is also possible to allow the sensors to be synchronized among each other, not via the main apparatus 50, by connecting the data transmission units 44, S44, S54, S64 using a parallel port, and by outputting a synchronous signal from the data transmission unit of one sensor to the data transmission unit of another sensor.

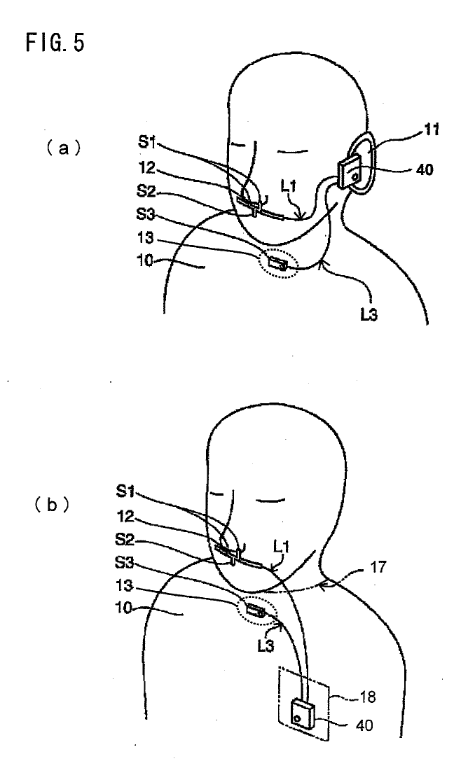

Before sleep, an examinee 10 attaches selected at least two of the synchronized sensors S1 to S6 at appropriate positions of the body so as to sense sleep data. As shown in FIGS. 5(a) and 5(b), the sensors S1 and S2 are integrated and attached to a portion 12 under the nose of the examinee 10, and connected to the recorder 40 via a common wire L1. Further, the sensor S3 is attached to a portion 13 of the throat of the examinee 10, and connected to the recorder 40 via a wire L3. It can also be designed so that the sensor S1 can separately measure breaths through the left and right nostrils.

The recorder 40 is attached to an ear 11 of the examinee 10 as shown in FIG. 5(a), or is stored in a pocket 18 of clothes 17 as shown in FIG. 5(b). The recorder 40 can also be attached to a shoulder of clothes (not shown).

Since the sensors S1 and S2 are attached to the portion 12 under the nose of the examinee 10, it is desirable to reduce the size of these sensors so as not to impede breathing of the examinee 10. As in the present embodiment, the integration of the sensors S1 and S2 makes it possible to reduce the size of the sensors as compared with the case where they are separate. Further, the sensors S1 to S3 are all placed at short distances around the face of the examinee 10. Thus, the sensors can be further reduced in size by allowing the supply of power and storage of data to rely on one recorder 40 than by allowing each sensor to individually have a built-in battery and storage unit.

In addition, the recorder 40 comprises e.g. a battery and a storage unit 42 for sensing data, so that multiple long wires from outside as in a conventional test equipment are not necessary, thereby reducing the complexity of wires. This makes it possible to provide good wearing comfort, and to effectively prevent a sensor from falling off even when the examinee 10 moves its face. Although the wires L1, L3 exist to connect the sensors S1 to S3 to the recorder 40, it imposes little burden on the examinee 10, because the distance between the sensors S1 to S3 and the recorder 40 is short, and the wires L1, L3 are short.

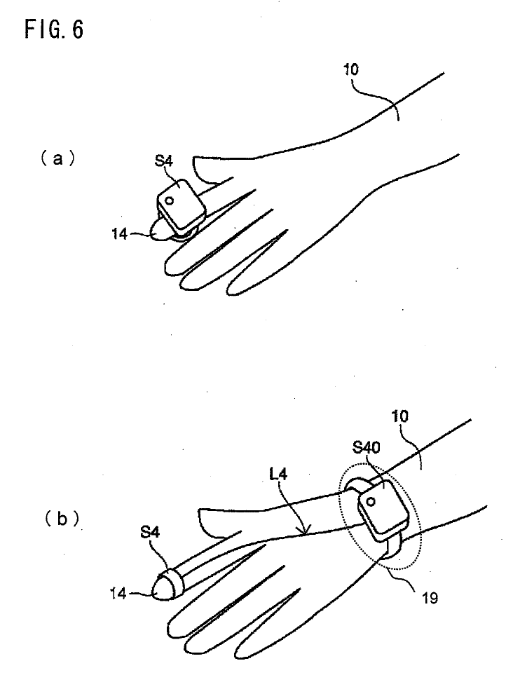

As shown in FIG. 6(a), the light sensor S4 for measuring blood oxygen concentration is attached to the tip of an index finger 14 of the examinee 10. Since this sensor S4 comprises its own battery and storage unit, long wires from outside as in the conventional test equipment are not necessary, so that it can be used independently. This makes it possible to provide good wearing comfort, and to effectively prevent the sensor from falling off even when the examinee 10 moves its hand.

As shown in FIG. 6(b), it is also possible to form the sensor S4 by only the sensing section, and separately provide a recorder S40 to be attached to a wrist 19 of the examinee 10 with a wire L4 connected therebetween. This more effectively reduces falling off of the sensor even when the examinee 10 violently moves the hand during test.

Next, referring again to FIG. 2, the acceleration sensor S5 for measuring chest movement and the acceleration sensor S6 for measuring stomach movement will be described. The former sensor S5 is attached near the chest portion 15 of the examinee 10, while the latter sensor S4 is attached near the stomach portion 16. Since these sensors S5, S6 also have their own battery and storage unit, they can respectively be used independently of each other, similarly as in the sensor S4. This makes it possible to provide good wearing comfort, and to effectively reduce falling off of the sensors even when the examinee 10 changes its posture during test.

The sensors S1 to S6, which are properly placed as described above, sense sleep data at certain preset frequencies, and the obtained sensing data are associated with the time data of the respective clock units and stored in the respective storage units. Here, the sensing frequencies of the respective sensors S1 to S6 are not necessary to be the same. Since the sensing period of each respective sensor is constant, it is sufficient if the sensing start time data is definite. For example, if the sensing frequency of the sensor S3 is 1 kHz, and the sensing frequency of the sensors S5, S6 is 50 Hz, then 1 data of the sensors S5, S6 corresponds to 20 data of the sensor S3. Similarly, if the sensing frequency of the sensors S1, S2 is 2 kHz, while that of the sensor S3 is 1 kHz, and that of the sensor S4 is 10 Hz, and further that of the sensors S5, S6 is 50 Hz, then the sensing frequencies of the sensors S3 to S6 other than the sensors S1, S2 are integer multiple of the sensing frequency of the sensors S1, S2. Accordingly, when all the sensors S1 to S6 are simultaneously synchronized by the main apparatus 50, the correlations between the respective sensing data and the time data can be made the same by calculating the relative ratios of the respective sensing frequencies, even if the sensing frequencies of the respective sensors S1 to S6 are not the same.

After sensing the sleep data, the sensors S1 to S6 and the recorder 40 are stored in the main apparatus 50. While thus stored, the sensing data and the time data stored in the respective storage units are transferred to the storage unit 52 of the main apparatus 50 via the respective data transmission units by the user operating the operation unit 54 to input a data transfer command. Further, in a medical institution, the main apparatus 50 is connected to the summing unit 60 via the network connection unit 56. Finally, the respective sensing data and the time data are transferred as a whole to the summing unit 60.

The sensing data and the time data having been transferred to the summing unit 60 are analyzed to diagnose SAS symptoms. The time data in the respective storage units corresponding to the sensors S1 to S6 are all based on the synchronous signal of the main apparatus 50, so that even when the sensors S1 to S6 are independently used without using transmission means such as e.g. wires and wireless signals, the synchronization between the sensing data of the sensors S1 to S6 is secured. Accordingly, the analysis of these sensing data makes it possible to have accurate sleep data of the examinee, and make accurate diagnosis of SAS.

Furthermore, in the present embodiment, the summing unit 60 performs data analysis which requires a summation of the sensing data and a complicated calculation process, so that it is sufficient if microcomputers e.g. provided in the control units of the recorder 40, the sensors S4 to S6 and the main apparatus 50 in the test equipment 30 have minimum required resources. This makes it possible to reduce the manufacturing cost and size of the equipment 30.

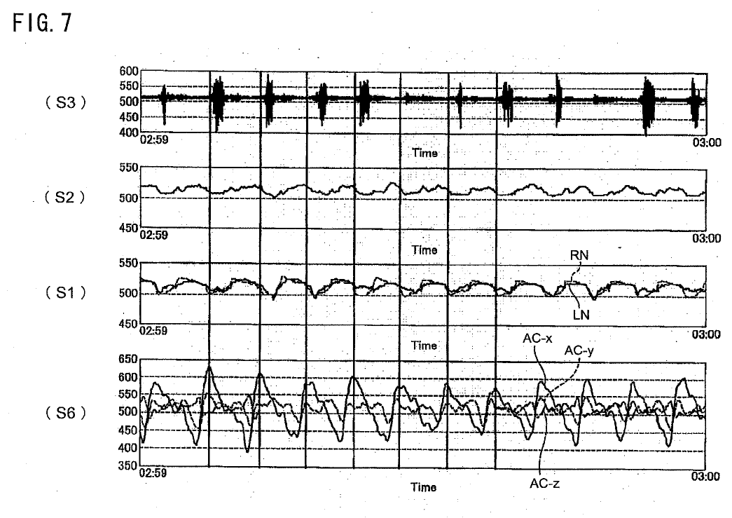

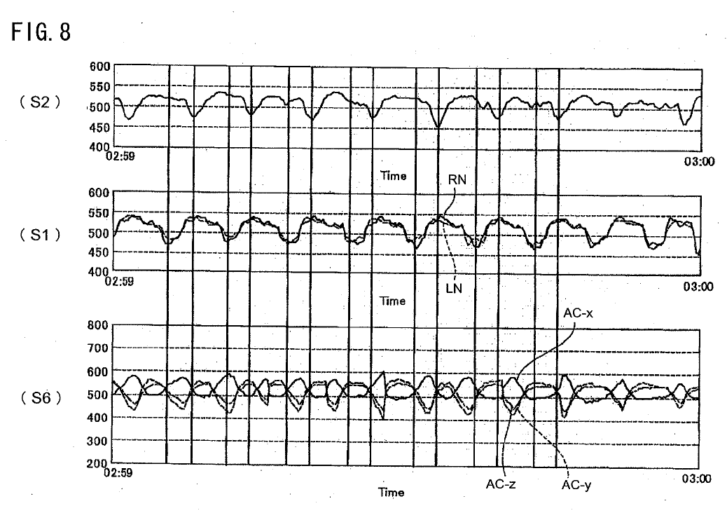

Next, sleep data measured by using the test equipment 30 as configured above will be described. FIG. 7 and FIG. 8 show examples of data obtained by measurements for the same examinee on different days, respectively. The measuring time zone of the data shown is 1 minute from 2:59 am to 3 am. The vertical lines connecting the respective graphs are auxiliary ones inserted to study the synchronization of the measured data. FIG. 7 shows sensing data when the examinee is in a sleep posture facing upward, while FIG. 8 shows sensing data when the examinee is in a sleep posture facing laterally.

S6 of FIG. 7 shows a time variation of a stomach movement based on the sensor S6. The upward direction of the vertical axis represents a direction in which the stomach expands, more specifically, the degree of inhalation, while the downward direction of the vertical axis represents a direction in which the stomach shrinks, more specifically, the degree of exhalation. The vertical auxiliary lines are inserted according to the peak values of inhalation of the stomach movement. Note that the acceleration sensors S5, S6 for measuring the stomach and chest movements can measure three-dimensional acceleration components, and that in the data shown by S6 of FIG. 7, AC-x denotes the direction from the head to the legs of the examinee when in a sleep posture facing upward, and AC-y the direction from the right to the left side of the body, while AC-z the direction from the back to the stomach.

S3 of FIG. 7 shows a time variation of snore based on the sensor S3. By comparing S6 and S3 of FIG. 7, it is seen that a snore occurs immediately after the stomach movement shows a peak value of inhalation.

S2 of FIG. 7 and S1 of FIG. 7 show time variations of mouth breath based on the sensor S2, and of nose breath based on the sensor S1, respectively. The upward direction of the vertical axis of S2 and S1 of FIG. 7 represents the degree of exhalation (temperature rise), while the downward direction of the vertical axis represents the degree of inhalation (temperature drop). Note that in the data shown by S1 of FIG. 7, RN denotes the breath of the right nostril, while LN denotes the breath of the left nostril. By comparing S6 and S2 of FIG. 7, it is seen that the inhalation of the nose breath is substantially synchronized with the timing of inhalation of the stomach movement. Further, by comparing S2 and S3 of FIG. 7, it is seen that the mouth breath, though small in degree, is substantially synchronized with the occurrence of snore. From these data, it is determined that the breath is normally taken although the examinee makes a snore after an inhalation.

On the other hand, S2 of FIG. 8 and S1 of FIG. 8 show time variations of mouth breath and nose breath, respectively. These S2 and S1 show that a peak of inhalation of a mouth breath occurs with a small delay from a peak of inhalation of a nose breath. From this, it is presumed that the examinee is taking not only nose breath but also mouth breath, and that the inside of the oral cavity and the laryngeal region are likely to be dry. Note that the data shown by S6 of FIG. 8 are data of the chest movement when the examinee changes in posture to face laterally, and show different waveforms from those of the stomach movement shown in FIG. 7. It is shown that the respective x, y, z components shown by the chest movement in FIG. 8, although different from each other in direction of change, periodically change corresponding to the breath.

In the actual diagnosis of SAS, the data obtained from ones of the sensors S1 to S6 are comprehensively analyzed by the summing unit 60 to count the occurrence frequency of apnea states of at least 10 seconds, so that as described in the foregoing, in the present invention, the correlation in time between the respective data is important, while at the same time it is important to allow the test equipment 30 to have such a structure as to reduce the burden on the examinee 10 to attach the sensors S1 to S6 and the recorder 40, thereby preventing disturbance of the sleep of the examinee 10, and as to secure the synchronization in time between the respective sensors, thereby making it possible to obtain accurate data. The present invention is not necessarily limited to the structure of the embodiment described above.

1. A sleep apnea test sensor assembly comprising multiple sensors to be used for diagnosis of sleep apnea syndrome, wherein:
the multiple sensors each comprise: a sensing section; a clock unit; a storage unit for storing sensing data measured by the sensing section and associated with time data of the clock unit; and a control unit for allowing the sensing section to perform sensing at a predetermined period in response to a clocking operation of the clock unit, and for setting time of the clock unit in response to a synchronous signal input from outside; and
the multiple sensors are comprised of a combination of at least two of a temperature sensor for measuring nose breath, a temperature sensor for measuring mouth breath, an acoustic sensor for measuring snore, a light sensor for measuring blood oxygen concentration, and acceleration sensors for measuring chest and stomach movements.

2. A sleep apnea test equipment comprising:
a sleep apnea test sensor assembly according to claim 1; and
a main apparatus having a storage section for storing the multiple sensors forming the sensor assembly, and having a control unit for outputting a synchronous signal to control time of the multiple sensors,
wherein the main apparatus collects the sensing data, together with the time data, of the respective sensors while stored in the storage section.
